(19) United States
(12) Patent Application Publication
(10) Pub. No.: US 2006/0131172 A1
(43) Pub. Date: Jun. 22, 2006

(54) Method of vertically aligning carbon nanotubes using electrophoresis

(76) Inventors: Ha-Jin Kim, Suwon-si (KR); Yong-Wan Jin, Seoul (KR); In-Taek Han, Seoul (KR); Hang-Woo Lee, Suwon-si (KR)

Correspondence Address: Robert E. Bushnell, Suite 300, 1522 K Street, N.W., Washington, DC, 20005, US

(21) Appl. No.: 11/294,399

(22) Filed: Dec. 6, 2005

(30) Foreign Application Priority Data
Dec. 18, 2004 (KR) 10-2004-0108415

(51) Int. Cl. C07K 1/26; C25B 7/00

(52) U.S. Cl. 204/450; 204/471

(57) ABSTRACT

A method of vertically aligning carbon nanotubes, whereby carbon nanotubes are grown on a substrate on which a catalyst metallic layer is formed, the grown carbon nanotubes are separated from the substrate in a bundle shape, the separated carbon nanotube bundles is put in an electrolyte having a charger, the carbon nanotube bundles are mixed with the charger to charge the carbon nanotube bundles, and the charged carbon nanotube bundles are vertically attached onto a surface of an electrode, using electrophoresis.

### CROSS-REFERENCE TO RELATED PATENT APPLICATIONS AND CLAIM OF PRIORITY

This application claims the benefit of Korean Patent Application No. 10-2004-0108415, filed on Dec. 18, 2004, in the Korean Intellectual Property Office, the disclosure of which is incorporated herein in its entirety by reference.

### BACKGROUND OF THE INVENTION

1. Field of the Invention

The present invention relates to a method of aligning carbon nanotubes (CNTs), and more particularly, to a method of vertically aligning carbon nanotubes (CNTs) using electrophoresis.

2. Description of the Related Art

Carbon nanotubes (CNTs) have been used in a variety of elements such as a field emission display (FED), a back-light for a liquid crystal display (LCD), a nanoelectronic device, an actuator, and a battery etc., since unique structural and electrical characteristics of CNTs have been known.

An FED is a display device which emits electrons from an emitter formed on a cathode, and emits light by a collision of the electrons with a phosphor layer formed on an anode. In these days, carbon nanotubes (CNTs) having high electron-emitting characteristics have been widely used as an emitter for an FED. An FED using CNTs as an emitter has a wide view angle, high resolution, low power, and high temperature stability etc., and thus can be used in a variety fields such as a view finer etc. for a car navigation apparatus or an electronic image apparatus. In particular, an FED can be used as a replaceable display apparatus in a personal computer (PC), a personal data assistants (PDA) terminal, a medical apparatus, or a high definition television (HDTV) etc.

In order to manufacture an FED having higher performance, CNTs used as an emitter should have a low driving voltage and a high emission current. To this end, CNTs should be vertically aligned on a cathode. That is, an emission current varies according to its alignment state even though CNTs have the same composition. Thus, in order to increase an emission current, it is preferable that as many as CNTs should be vertically aligned on the cathode.

### SUMMARY OF THE INVENTION

The present invention provides a method of vertically aligning carbon nanotubes (CNTs) that have been vertically grown at a high temperature, using electrophoresis at a low temperature.

According to an aspect of the present invention, there is provided a method of vertically aligning carbon nanotubes, the method including: growing carbon nanotubes on a substrate on which a catalyst metallic layer is formed; separating the grown carbon nanotubes from the substrate in a bundle shape; putting the separated carbon nanotube bundles in an electrolyte having a charger, and mixing the carbon nanotube bundles with the charger to charge the carbon nanotube bundles; and vertically attaching the charged carbon nanotube bundles onto a surface of an electrode, using electrophoresis.

Catalyst metallic particles may be attached on both-ends of the grown carbon nanotubes. The charger may be mixed with the catalyst metallic particles attached on both-ends of the carbon nanotubes and may charge the both-ends of the carbon nanotube bundles to positive (+).

When a predetermined voltage is applied between a pair of electrodes provided in the electrolyte, one end of the carbon nanotube bundles charged to positive (+) may be attached onto a surface of a cathode of the pair of electrodes.

In this case, a direct current or an alternating current may be applied between the pair of electrodes.

The catalyst metallic layer may be formed by depositing a predetermined catalyst metal on the substrate. In addition, the catalyst metallic layer may be formed by depositing a predetermined catalyst metal on the substrate and by patterning the deposited catalyst metal in a predetermined shape.

The catalyst metallic layer may be formed of at least one metal selected from the group consisting of Fe, Ni, and Co.

The carbon nanotubes may be vertically grown on the catalyst metallic layer using CVD. A metallic thin film may be deposited on upper ends of the carbon nanotubes that have been grown on the substrate.

The carbon nanotubes that have been grown on the catalyst metallic layer may be separated from the substrate in a bundle shape using ultrasonic waves, and the carbon nanotube bundles put in the electrolyte may be mixed with the charger using ultrasonic waves.

### BRIEF DESCRIPTION OF THE DRAWINGS

A more complete appreciation of the present invention, and many of the above and other features and advantages of the present invention, will be readily apparent as the same becomes better understood by reference to the following detailed description when considered in conjunction with the accompanying drawings in which like reference symbols indicate the same or similar components, wherein:

FIGS. 1 through 6 illustrate methods of vertically aligning carbon nanotubes (CNTs) according to embodiments of the present invention;

FIG. 7 is a photo showing CNTs grown on a substrate on which a catalyst metallic layer is formed, using thermal chemical vapor deposition (CVD);

FIGS. 8 and 9 are photos showing catalyst metallic particles attached on both-ends of the grown CNTs;

FIG. 10 is a photo showing a catalyst metallic layer patterned on the substrate and CNTs grown on the catalyst metallic layer; and

FIGS. 11 and 12 are photos showing carbon nanotube (CNT) bundles that have been vertically aligned on a cathode.

### DETAILED DESCRIPTION OF THE INVENTION

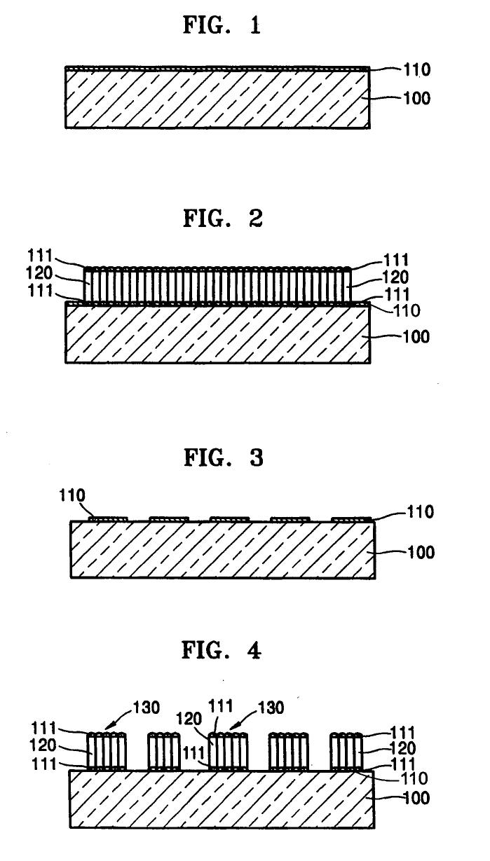

FIGS. 1 through 6 illustrate methods of vertically aligning carbon nanotubes (CNTs) according to embodiments of the present invention. Referring to FIG. 1, a metallic layer 110 is formed on a substrate 100. Specifically, a predetermined catalyst metal is deposited on the substrate 100, using magnetron sputtering or e-beam evaporation, thereby forming a catalyst metallic layer 110 on which CNTs can be grown. Here, the catalyst metal may be at least one metal selected from the group consisting of Fe, Ni, and Co.

Referring to FIG. 2, carbon nanotubes (CNTs) 120 are vertically grown on the catalyst metallic layer 110, using chemical vapor deposition (CVD). Here, the CNTs 120 may be grown using thermal CVD or plasma enhanced CVD (PE CVD). Specifically, in CNTs growth using thermal CVD, the growth uniformity of CNTs is very high and CNTs having a smaller diameter than in PE CVD can be grown such that CNTs having a low turn on voltage can be formed. In CNTs growth using PE CVD, CNTs can be grown to be more perpendicular to a substrate, and synthesis can be performed at a lower temperature, compared with CNTs growth using thermal CVD. Vertical growth of CNTs depends on the direction of an electric field applied between an anode and a cathode in a PE CVD system. Thus, the growth direction of CNTs can be adjusted according to the direction of the electric field. In addition, since the growth direction of CNTs is uniform, the density of CNTs can be easily adjusted and electrons can be easily emitted by an electric field.

If the CNTs 120 are vertically grown on the catalyst metallic layer 110 formed on the substrate 100 using CVD in this way, catalyst metallic particles 111 which may come from the catalyst metallic layer are attached on each of both-ends of the grown CNTs 120.

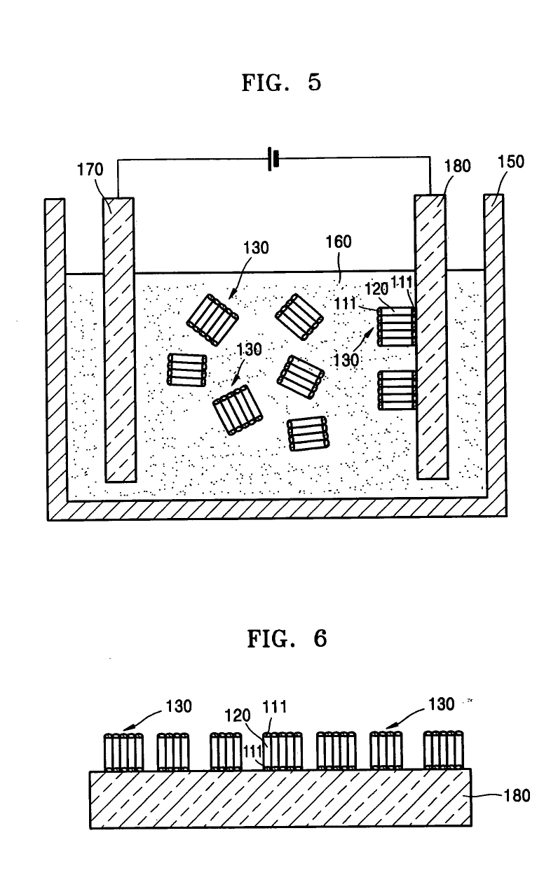

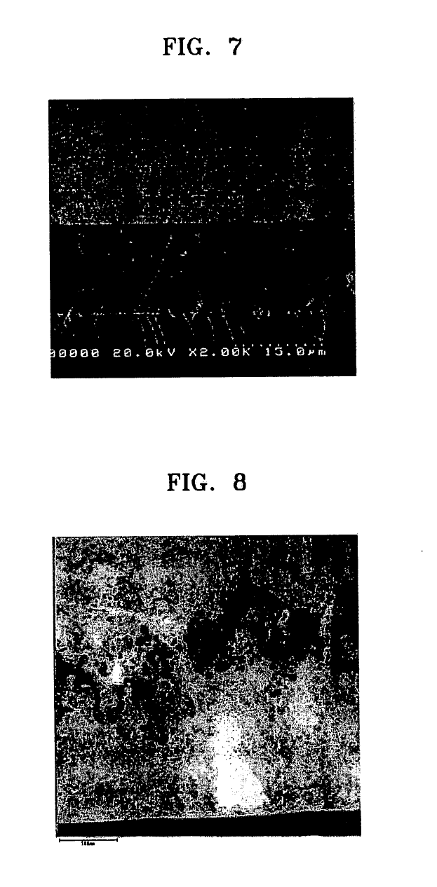

FIG. 7 is a photo showing that the CNTs are vertically grown on the catalyst metallic layer formed on the substrate. FIGS. 8 and 9 illustrate enlarged plane view and cross-section of the CNTs shown in FIG. 7. FIGS. 8 and 9 show that the catalyst metallic particles (black portion) are attached on both-ends of the CNTs that have been vertically grown on the catalyst metallic layer formed on the substrate. A metallic thin film (not shown) may be deposited on an upper end of the CNTs 120 SO that the CNTs 120 can be easily attached to a cathode 180 by an electric field applied into an electrolyte (160 of FIG. 5) that will be described later.

The CNTs 120 that have been vertically grown on the substrate 100 in this way are separated from the substrate 100 in a bundle shape, preferably using ultrasonic waves. Here, if ultrasonic waves are applied to the CNTs 120 and the substrate 100 for about 2 to 3 minutes, the CNTs 120 can be separated from the substrate 100 in a bundle shape.

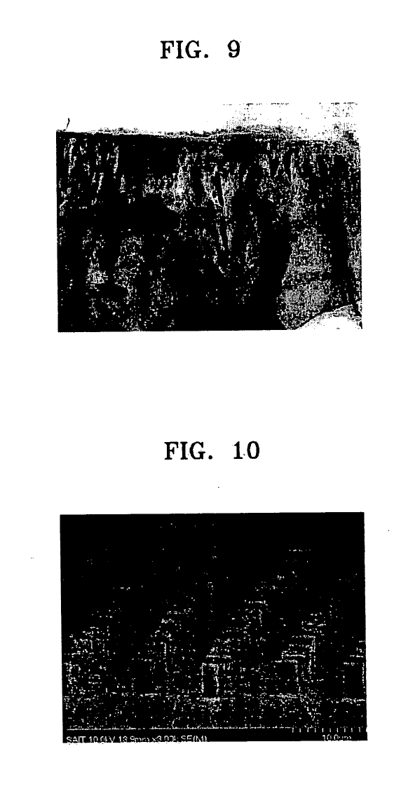

The CNTs 120 may be formed on the catalyst metallic layer 110 patterned on the substrate 100 in a bundle shape. Specifically, referring to FIG. 3, the catalyst metallic layer 110 patterned in a predetermined shape is formed on the substrate 100. Here, the patterned catalyst metallic layer 110 may be formed by depositing a predetermined catalyst metal on the surface of the substrate 100 and by patterning the deposited catalyst metal in a predetermined shape, for example, in a dot shape. Referring to FIG. 4, the CNTs 120 may be grown on the patterned catalyst metallic layer 110, using CVD described above. As such, carbon nanotube (CNT) bundles 130 are vertically grown on the patterned catalyst metallic layer 110. The catalyst metallic particles 111 are also attached on both-ends of the CNTs 120 that have been grown in the above-described bundle shape. FIG. 10 is a photo showing the CNT bundles which are grown on the catalyst metallic layer patterned on the substrate. The above-described metallic thin film may be deposited on upper ends of the CNT bundles 130.

Subsequently, the CNT bundles 130 formed on the catalyst metallic layer 110 patterned on the substrate 100 are separated from the substrate 100, using ultrasonic waves. In this way, if the catalyst metallic layer 110 patterned on the substrate 100 is formed and the CNT bundles 130 are formed on the catalyst metallic layer 110 and separated from the substrate 100, the CNT bundles 130 formed of a predetermined number of carbon nanotubes 120 can be obtained.

Referring to FIG. 5, the CNT bundles 130 separated from the substrate 100 are put in an electrolyte 160 filled in a container 150. Here, the electrolyte 160 may be isopropyl alcohol (IPA). A charger (not shown) having a positive (+) charge is included in the electrolyte 160. Examples of the charger having a positive (+) charge include Mg(NO₃)₂ and Al(NO₃)₂, but are not limited thereto. A pair of electrodes 170 and 180 are provided in the electrolyte 160. Subsequently, the CNT bundles 130 put in the electrolyte 160 and the charger are mixed with each other, thereby charging the CNT bundles 130 to positive (+). Specifically, if ultrasonic waves are applied to the CNT bundles 130 and the electrolyte 160 in which the charger is included for a predetermined amount of time, the charger is mixed with the catalyst metallic particles 111 attached on both-ends of the CNT bundles 130, thereby charging both-ends of the CNT bundles 130 to positive (+). Next, the CNT bundles 130 are vertically attached onto the surface of one electrode 180 of the pair or electrodes 170 and 180, using electrophoresis. Specifically, if a predetermined voltage, for example, about 25 to 35V, preferably, about 30V is applied between the pair of electrodes 170 and 180, an electric field is formed between the pair of electrodes 170 and 180, and owing to formation of the electric field, one end of the CNT bundles 130 charged to positive (+) is attached onto the surface of the cathode 180 of the pair of electrodes 170 and 180 having a cathode 180 and an anode 170. As such, the CNT bundles 130 are vertically attached onto the surface of the cathode 180. Here, a current that flows between the pair of electrodes 170 and 180 in the electrolyte 160 may be about 5 to 10 mA. An alternating current (AC) voltage as well as a direct current (DC) voltage may be applied between the pair of electrodes 170 and 180.

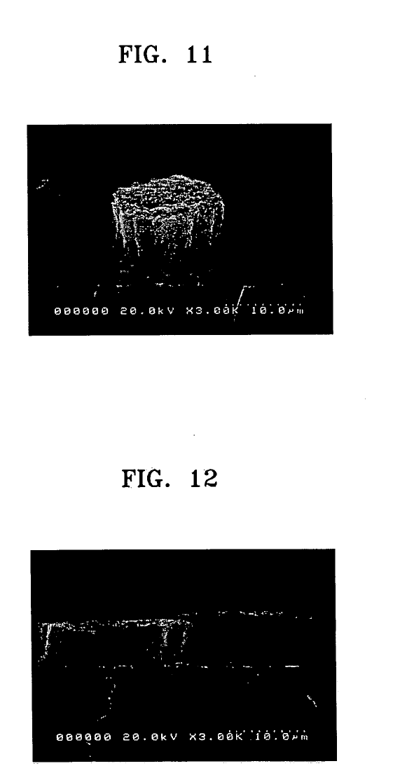

If the CNT bundles 130 are attached onto the surface of the cathode 180, using electrophoresis, as shown in FIG. 6, the CNT bundles 130 that have been vertically aligned on the cathode 180 can be obtained. FIGS. 11 and 12 are photos in which CNT bundles are attached onto the surface of the cathode using electrophoresis. FIGS. 11 and 12 show that the CNT bundles are vertically aligned on the surface of the cathode.

As described above, in the method of vertically aligning carbon nanotubes (CNTs) according to the present invention, CNTs that have been vertically grown at a high temperature are self assembled on the surface of an electrode at a low temperature using electrophoresis such that the CNTs can be vertically aligned on the electrode. As such, an array of CNTs with a good quality that has been vertically and well aligned can be manufactured.

While the present invention has been particularly shown and described with reference to an exemplary embodiment thereof, it will be understood by those skilled in the art that various changes in form and details may be made therein without departing from the spirit and scope of the invention as defined by the following claims.

What is claimed is:

1. A method of vertically aligning carbon nanotubes, the method comprising:
growing carbon nanotubes on a substrate on which a catalyst metallic layer is formed;
separating the grown carbon nanotubes from the substrate in a bundle shape;
putting the separated carbon nanotube bundles in an electrolyte having a charger and mixing the carbon nanotube bundles with the charger to charge the carbon nanotube bundles; and
vertically attaching the charged carbon nanotube bundles onto a first electrode in the electrolyte by using electrophoresis.

2. The method of claim 1, wherein catalyst metallic particles are attached on both-ends of the grown carbon nanotubes.

3. The method of claim 2, wherein the charger is mixed with the catalyst metallic particles attached on both-ends of the carbon nanotubes and charges the both-ends of the carbon nanotube bundles to positive (+).

4. The method of claim 3, wherein the vertical attachment of the charged carbon nanotube bundles onto the first electrode comprises applying a predetermined voltage between the first electrode and a second electrode provided in the electrolyte to attach one end of the carbon nanotube bundles charged to positive (+) onto the first electrode.

5. The method of claim 4, wherein a direct current or an alternating current is applied between the pair of electrodes.

6. The method of claim 5, wherein the predetermined voltage ranges from 25 to 35V.

7. The method of claim 6, wherein a current that flows between the pair of electrodes is 5 to 10 mA.

8. The method of claim 1, wherein the catalyst metallic layer is formed by depositing a predetermined catalyst metal on the substrate and patterning the deposited catalyst metal in a predetermined shape.

9. The method of claim 1, wherein the catalyst metallic layer is formed of at least one metal selected from the group consisting of Fe, Ni, and Co.

10. The method of claim 1, wherein the carbon nanotubes are vertically grown on the catalyst metallic layer by chemical vapor deposition.

11. The method of claim 1, further comprising depositing a metallic film on upper ends of the carbon nanotubes grown on the substrate.

12. The method of claim 1, wherein the separation of the grown carbon nanotubes is performed by using ultrasonic waves.

13. The method of claim 1, wherein the electrolyte is isopropyl alcohol (IPA).

14. The method of claim 1, wherein the carbon nanotube bundles put in the electrolyte are mixed with the charger using ultrasonic waves.

15. A method of vertically aligning carbon nanotubes, the method comprising:
growing carbon nanotubes on a catalyst metallic layer formed on a substrate;
separating the grown carbon nanotubes from the substrate in a bundle shape;
charging the carbon nanotubes to positive (+); and
performing electrophoresis to vertically attach the charged carbon nanotube bundles onto an electrode.

16. The method of claim 15, wherein the catalyst metallic layer is patterned on the substrate.

17. The method of claim 15, wherein the carbon nanotubes are charged to positive (+) by putting the carbon nanotubes in an electrolyte having a charger and mixing the carbon nanotubes with the charger.

18. The method of claim 15, further comprising depositing a metallic film on upper ends of the grown carbon nanotubes on the substrate.

19. A method of vertically aligning carbon nanotubes, the method comprising:
growing carbon nanotubes on a substrate on which a catalyst metallic layer is formed;
putting the separated carbon nanotube bundles in a container having an electrolyte and a charger;
charging the carbon nanotube bundles with the charger; and
applying a predetermined voltage between a first electrode and a second electrode positioned in the electrolyte to attach one end of the carbon nanotube bundles onto the first electrode.

20. The method of claim 19, wherein the catalyst metallic layer is patterned on the substrate.
